(19) United States
(12) Patent Application Publication
(10) Pub. No.: US 2024/0099554 A1
(43) Pub. Date: Mar. 28, 2024

(54) MEDICAL DEVICES AND RELATED SYSTEMS AND METHODS FOR AUTOMATIC IMAGE BRIGHTNESS CONTROL

(71) Applicant: Boston Scientific Scimed, Inc., Maple Grove, MN (US)

(72) Inventors: Louis J BARBATO, Franklin, MA (US); Kirsten VIERING, Newton, MA (US)

(73) Assignee: Boston Scientific Scimed, Inc., Maple Grove, MN (US)

(21) Appl. No.: 18/472,328

(22) Filed: Sep. 22, 2023

Related U.S. Application Data
(60) Provisional application No. 63/377,433, filed on Sep. 28, 2022.

(51) Int. Cl. A61B 1/00; A61B 1/06; H04N 23/71; H04N 23/73; H04N 23/74; H04N 23/76

(52) U.S. Cl. CPC A61B 1/00006; A61B 1/00009; A61B 1/00105; A61B 1/0655; H04N 23/71; H04N 23/73; H04N 23/74; H04N 23/76

(57) ABSTRACT

A medical device system may include a control unit comprising one or more processors that implement an algorithm to enhance images obtained by a medical device. The one or more processing boards perform the steps of: receiving a first image from the first viewing element; determining a current illumination value of the first image; determining a first difference between the current illumination value and a target high illumination value if the current illumination value is greater than the target high illumination value; determining a second difference between the current illumination value and a target low illumination value if the current illumination value is less than the target low illumination value; generating a new illumination value, using at least one of the first difference and the second difference; and converting the new illumination value to a first voltage value for application to one or more illuminators of the medical device.

### CROSS-REFERENCE TO RELATED APPLICATIONS

This application claims the benefit of priority from U.S. Provisional Application No. 63/377,433, filed Sep. 28, 2022, which is incorporated by reference herein in its entirety.

### TECHNICAL FIELD

Various aspects of this disclosure relate generally to systems, devices, and methods for automatic image brightness control. More specifically, embodiments of this disclosure relate to imaging catheters, such as endoscope or other medical devices, configured to automatically control an illuminator and related systems and methods, among other aspects.

### BACKGROUND

During endoscopic procedures, a medical professional operating an endoscope often relies on one or more illuminators to illuminate a field of view of a camera at the distal end of the endoscope. Most imaging catheters, such as endoscopes, rely on a fixed illumination output, with each of the imaging catheter's illuminators outputting a constant illumination, for example, from one or more light emitting diodes (LEDs). Such imaging catheters with a constant illumination output control the image brightness by varying the exposure and/or the gain of the image sensor. This results in a noticeable stepped response in the image brightness, as well as a slow response to changing scenes. Step response refers to the change of the output of a system when its input is a unit step function. The stepped response is due to the limited number of exposure steps available in the image sensor, and the slowed response is at least in part because the exposure values are written in single steps at the end of each image frame. For example, if the exposure needs to be adjusted by 10 steps to increase or decrease the exposure of the image sensor, then it would typically take a minimum of 10 image frames to adjust the brightness of the image, resulting in a noticeable lag (approximately 300 milliseconds for a 30 frame per second (fps) image sensor).

When a user experiences image lag in an imaging catheter system, the procedure may be prolonged, and procedural tasks may be more difficult and delayed. There is a need for alternative methods of illumination adjustment for imaging catheters and other medical devices to reduce imaging lag and address other problems with medical device illumination and imaging systems.

### SUMMARY

Aspects of the disclosure relate to, among other things, systems, devices, and methods to help reduce imaging lag in medical device imaging systems, among other aspects. The systems, devices, and methods of this disclosure may decrease the time required to focus and/or properly illuminate a field of view of a camera or other imaging device of an endoscope or other medical device. Endoscopes and other medical devices incorporating the systems and methods of this disclosure may help address image lag, may help reduce the time required for procedures, and may help address other issues. Each of the aspects disclosed herein may include one or more of the features described in connection with any of the other disclosed aspects.

According to one aspect, a medical device system may include a control unit configured to be operatively coupled to a medical device. The control unit may comprise one or more processors that implement an algorithm to enhance images obtained by a first viewing element of the medical device. The one or more processing boards perform the steps of: receiving a first image from the first viewing element; determining a current illumination value of the first image; determining a first difference between the current illumination value and a target high illumination value if the current illumination value is greater than the target high illumination value; determining a second difference between the current illumination value and a target low illumination value if the current illumination value is less than the target low illumination value; generating a new illumination value, using at least one of the first difference and the second difference; and converting the new illumination value to a first voltage value for application to one or more illuminators of the medical device.

In other aspects, the medical device system may include one or more of the following features. The target high illumination value and the target low illumination value together may define a tolerance band around a target illumination value stored by the control unit. The one or more processing boards may further perform the steps of: determining if the current illumination value is below a first threshold illumination value; and if the current illumination value is below the first threshold illumination value, using a scaling factor to generate the new illumination value. The one or more processing boards may further perform the steps of: determining if the new illumination value is greater than a maximum illumination value; if the new illumination value is greater than a maximum illumination value, converting the maximum illumination value to a second voltage value for application to one or more illuminators of the medical device, and increasing a gain of the one or more imaging devices. The one or more processing boards may further perform the steps of: determining if the new illumination value is lower than the current illumination value; if the new illumination value is lower than the current illumination value, decreasing a gain of the one or more imaging devices. The medical device may be an endoscope. Determining the current illumination value of the first image may include accumulating and summing pixel values of the first image. The one or more processing boards may further perform the steps of: determining a current frame rate of the first viewing element; generating a new frame rate, using at least one of the first difference and the second difference; and applying the new frame rate to the first viewing element.

In other aspects, the medical device system may include one or more of the following features. The medical device may include a first viewing element and at least one illuminator. The one or more processors may further perform the steps of: determining a current exposure time of the first viewing element; generating a new exposure time, using at least one of the first difference and the second difference; and applying the new exposure time to the first viewing element. The one or more processors may further perform the steps of: prior to converting the new illumination value to the first voltage value, determining if the current illumination value is below or above the new illumination value; if the current illumination value is below the new illumination value, converting the new illumination value to the first voltage value; and if the current illumination value is above the new illumination value, increasing a frame rate of the first viewing element. Generating a new illumination value, using at least one of the first difference and the second difference, may include determining a first error coefficient of the first image and a second error coefficient of a second image, wherein the second image was received by the control unit prior to the first image; wherein the first error coefficient is the first difference if the current illumination value is greater than the target high illumination value; and wherein the first error coefficient is the second difference if the current illumination value is less than the target low illumination value. Generating the new illumination value may further include determining a proportional tuning constant, an integral tuning constant, and a derivative tuning constant each associated with the medical device.

In other aspects, the medical device system may include one or more of the following features. The current illumination value may be a first current illumination value, the target high illumination value may be a first target high illumination value, the target low illumination value may be a first target low illumination value, the new illumination value may be a first new illumination value, and the one or more processing boards may further perform the steps of: receiving a second image from a second viewing element; determining a second current illumination value of the second image; determining a third difference between the second current illumination value and a second target high illumination value if the second current illumination value is greater than the second target high illumination value; determining a fourth difference between the second current illumination value and a second target low illumination value if the second current illumination value is less than the second target low illumination value; generating a second new illumination value, using at least one of the third difference and the fourth difference; and converting the second new illumination value to a second voltage value for application to one or more illuminators of the medical device. The one or more processing boards may further perform the steps of: displaying, via at least one electronic display, a second image received from the first viewing element, and the second image is illuminated by the one or more illuminators receiving the first voltage.

In other aspects, a method of enhancing images obtained by a medical device system is disclosed. The medical device system may comprise (a) one or more processers, and (b) a medical device operatively coupled to the one or more processers, wherein the medical device is configured to be inserted into a body of a patient and includes a first viewing element and one or more illuminators. The method comprising the steps of: receiving a first image from the first viewing element; determining a current illumination value of the first image; determining a first difference between the current illumination value and a target high illumination value if the current illumination value is greater than the target high illumination value; determining a second difference between the current illumination value and a target low illumination value if the current illumination value is less than the target low illumination value; generating a new illumination value, using at least one of the first difference and the second difference; and converting the new illumination value to a first voltage value for application to the one or more illuminators of the medical device.

In other aspects, the method may include one or more of the following features. The method may further comprise the steps of determining if the new illumination value is greater than a maximum illumination value; if the new illumination value is greater than a maximum illumination value, converting the maximum illumination value to a second voltage value for application to one or more illuminators of the medical device, and increasing a gain of the one or more imaging devices. The method may further comprise the steps of: determining a current exposure time of the first viewing element; generating a new exposure time, using at least one of the first difference and the second difference; and applying the new exposure time to the first viewing element. The method may further comprise the steps of: determining a current frame rate of the first viewing element; generating a new frame rate, using at least one of the first difference and the second difference; and applying the new frame rate to the first viewing element.

In other aspects, a non-transitory computer readable medium may contain program instructions for causing a computer to perform a method of enhancing images obtained by a first viewing element in a medical device system, and the medical device system may comprise a processor configured to implement the process, and a medical device operatively coupled to the processor, the medical device being configured for insertion into a body of a patient and including the first viewing element and one or more illuminators. The method may comprise the steps of: receiving a first image from the first viewing element; determining a current illumination value of the first image; determining a first difference between the current illumination value and a target high illumination value if the current illumination value is greater than the target high illumination value; determining a second difference between the current illumination value and a target low illumination value if the current illumination value is less than the target low illumination value; generating a new illumination value, using at least one of the first difference and the second difference; and converting the new illumination value to a first voltage value for application to the one or more illuminators of the medical device.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIGS. 1A and 1B are perspective views of an exemplary endoscope system, according to aspects of this disclosure.

FIG. 2 illustrates an exemplary method for automatically adjusting illumination in a medical device, according to aspects of this disclosure.

FIG. 3 illustrates an optional additional portion of the method of FIG. 2, according to aspects of this disclosure.

FIG. 4 illustrates another exemplary method for automatically adjusting illumination in a medical device, according to aspects of this disclosure.

FIG. 5 illustrates an exemplary chart of the illumination values applied to one or more illuminators and exposure time values applied to one or more imaging devices in a PID control system, according to aspects of this disclosure.

FIG. 6 illustrates another exemplary method for automatically adjusting illumination in a medical device, according to aspects of this disclosure.

FIG. 7 is a simplified functional block diagram of a computer and/or server that may be configured as a device or system performing any of the methods described herein, according to aspects of this disclosure.

### DETAILED DESCRIPTION

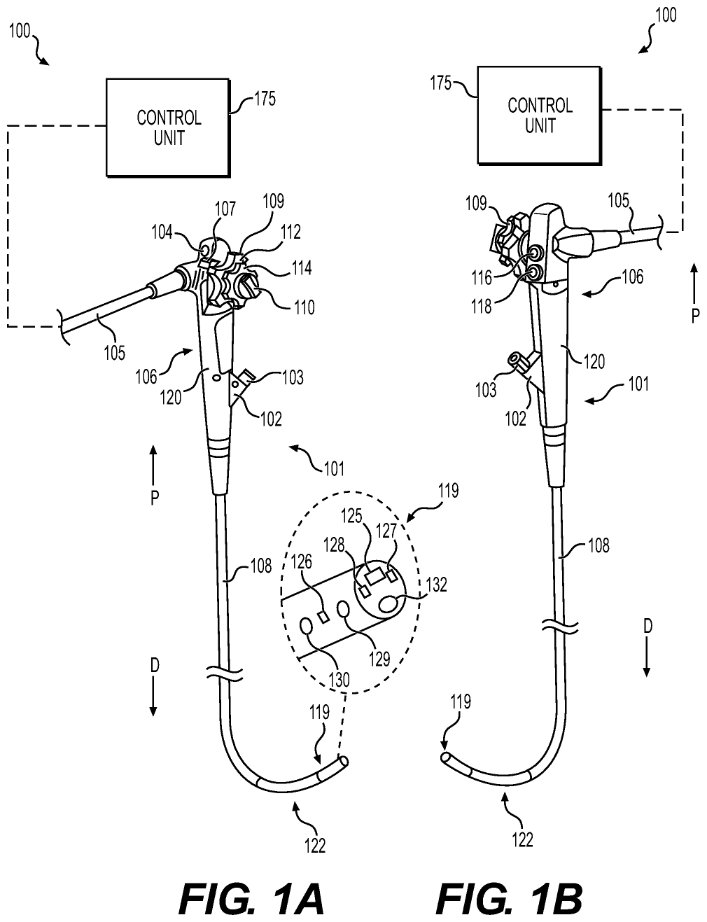

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. In FIGS. 1A and 1B, arrows labeled “P” and “D” are used to show the proximal and distal directions in the figure. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Embodiments of this disclosure seek to improve the illumination and imaging of a medical device, such as an endoscope, during a medical procedure. As non-limiting exemplary benefits, aspects of this disclosure may reduce the lag experienced with an imaging system and/or may facilitate viewing a field of view of one or more cameras of a medical device, among other aspects.

FIGS. 1A and 1B show perspective views of an exemplary endoscope system 100. Endoscope system 100 may include an endoscope 101. Although the term endoscope may be used herein, it will be appreciated that other devices, including, but not limited to, duodenoscopes, colonoscopes, ureteroscopes, bronchoscopes, laparoscopes, sheaths, catheters, or any other suitable delivery device or other type of medical device may be used in connection with the systems and methods of this disclosure, and the systems and methods discussed below may be incorporated into any of these or other medical devices.

Endoscope 101 may include a handle assembly 106 and a flexible tubular shaft 108. The handle assembly 106 may include one or more of a biopsy port 102, a biopsy cap 103, an image capture button 104, an elevator actuator 107, a locking lever 109, a locking knob 110, a first control knob 112, a second control knob 114, a suction button 116, an air/water button 118, a handle body 120, and an umbilicus 105. All of the actuators, elevators, knobs, buttons, levers, ports, or caps of endoscope system 100, such as those enumerated above, may serve any purpose and are not limited by any particular use that may be implied by the respective naming of each component used herein. The umbilicus 105 may extend from handle body 120 to auxiliary devices, such as a control unit 175, water/fluid supply, and/or vacuum source. Umbilicus 105 may transmit signals between endoscope 101 and control unit 175, in order to control lighting and imaging components of endoscope 101 and/or receive image data from endoscope 101. Umbilicus 105 also can provide fluid for irrigation from the water/fluid supply and/or suction to a distal tip 119 of shaft 108. Buttons 116 and 118 may control valves for suction and fluid supply (e.g., air and water), respectively. Shaft 108 may terminate at distal tip 119. Shaft 108 may include an articulation section 122 for deflecting distal tip 119 in up, down, left, and/or right directions. Knobs 112 and 114 may be used for controlling such deflection. Locking lever 109 and locking knob 110 may lock knobs 112 and 114, respectively, in desired positions.

Distal tip 119 may include one or more imaging devices 125, 126 and lighting sources 127-130 (e.g., one or more LEDs, optical fibers, and/or other illuminators). Examples of imaging devices (or viewing elements) 125, 126 include one or more cameras, one or more image sensors, endoscopic viewing elements, optical assemblies including one or more image sensors and one or more lenses, and any other imaging device known in the art. As shown in FIG. 1A, distal tip 119 may include a front-facing imaging device 125 and a side-facing imaging device 126. However, in other embodiments, distal tip 119 may include only one imaging device 125, 126, which may be front-facing or side-facing. In other examples, distal tip 119 may include three or more imaging devices 125, 126 directed in different directions, and in some examples the fields of view of each imaging device 125, 126 may overlap. Distal tip 119 may include one or more illuminators 127-130, and one or more illuminators 127, 128 may be front-facing illuminators (or face the distal direction), and one or more illuminators may be side-facing illuminators 129, 130. Side-facing imaging device 126 and side-facing illuminators 129, 130 may face radially outward, perpendicularly, approximately perpendicularly, or otherwise transverse to a longitudinal axis of shaft 108 and distal tip 119. Front-facing or forward-facing imaging device 125 and front-facing illuminators 127, 128 may face approximately along a longitudinal axis of distal tip 119 and shaft 108.

The disclosed endoscope system 100 may also include control unit 175, as depicted in FIGS. 1A and 1B. Control unit 175 may be capable of interfacing with endoscope 101 to provide power and instructions for imaging devices 125, 126 and illuminators 127-130. Control unit 175 may also control other aspects of endoscope 101, such as, for example, the application of suction, the deployment or delivery of fluid, and/or the movement of distal tip 119. Control unit 175 may be powered by an external source such as an electrical outlet. In addition, the control unit 175 may include buttons, knobs, touchscreens, or other user interfaces to control the imaging devices 125, 126, illuminators 127-130, and other features of endoscope 101. The control unit 175 may be housed in the handle body 120 itself or in a separate apparatus.

Control unit 175 may be configured to enable the user to set or control one or more illumination and imaging parameters. For example, control unit 175 may enable the user to set or control an illumination level for each of illuminators 127-130, gain level for each of the imaging devices 125, 126, exposure time for each of the imaging devices 125, 126, frame rate of each of the imaging devices 125, 126, maximum or target values for any of the illumination and imaging parameters, and/or any other parameter associates with the imaging devices 125, 126 and illuminators 127-130. In some examples, control unit 175 may be configured to execute one or more algorithms using one or more illumination and imaging parameters, for example to automatically adjust an illumination level of one or more of illuminators 127-130 and/or automatically adjust one or more parameters of imaging devices 125, 126. For example, control unit 175 may set or select an illumination level for one or more illuminators 127-130 based on data received from one or more imaging devices 125, 126.

Control unit 175 may include electronic circuitry configured to receive, process, and/or transmit data and signals between endoscope 101 and one or more other devices. For example, control unit 175 may be in electronic communication with a display configured to display images based on image data and/or signals processed by control unit 175, which may have been generated by the imaging devices 125, 126 of endoscope 101. Control unit 175 may be in electronic communication with the display in any suitable manner, either via wires or wirelessly. The display may be manufactured in any suitable manner and may include touch screen inputs and/or be connected to various input and output devices such as, for example, mouse, electronic stylus, printers, servers, and/or other electronic portable devices. Control unit 175 may include software and/or hardware that facilitates operations such as those discussed above. For example, control unit 175 may include one or more algorithms, models, or the like for executing any of the methods and/or systems discussed in this disclosure, and may be configured to automatically adjust the illumination value applied to one or more illuminators 127-130, and automatically adjust the gain and the frame rate applied to the one or more imaging devices 125, 126.

In operating endoscope system 100, a user may use his/her left hand to hold handle assembly 106 while the right hand is used to hold accessory devices and/or operate one or more of the actuators of the handle assembly 106, such as first and second control knobs 112, 114 and locking lever 109 and locking knob 110. When grasping handle body 120, the user may use a left-hand finger to operate image capture button 104, suction button 116, and/or air/water button 118 (each by pressing). During a procedure, a user may view the field of view of one or more of imaging devices 125, 126 on an electronic display operable connected to control unit 175. The one or more illuminators 127-130 may provide illumination to the field of view of the one or more of imaging devices 125, 126.

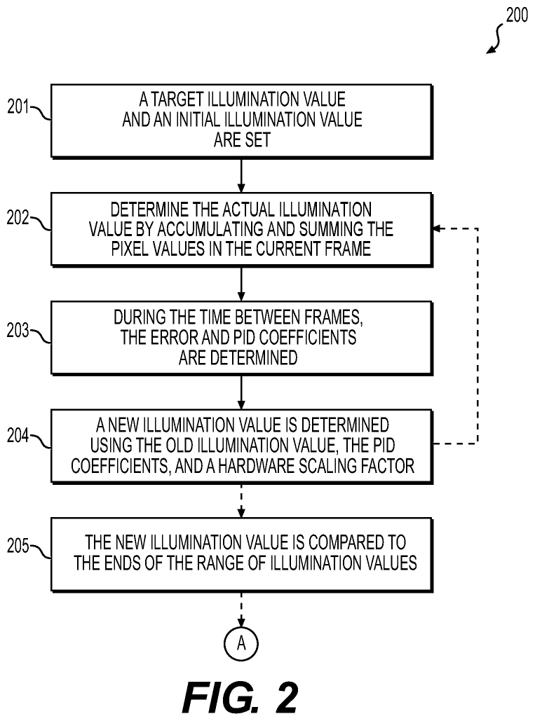

FIGS. 2-4 are process flow diagrams illustrating various control-loop algorithms that may be implemented by endoscope system 100 or any other medical device system with one or more imaging devices and one or more illuminators. Although the algorithms described herein are discussed in relation to an endoscope system, the algorithms are not so limited and may be implemented using any medical device system known in the art that includes imaging components and illumination components. In general, the algorithms discussed herein vary the illumination level based on the changing data received from the one or more imaging devices, for example the changing field of view of an imaging device.

FIG. 2 illustrates an illumination control method 200 that may be automatically executed by control unit 175 of endoscope system 100. Method 200 utilizes proportional, integral, and derivative (PID) coefficients to control the speed and accuracy of the illumination in endoscope system 100. At initial step 201, a target image brightness is stored in control unit 175 and an initial illumination value is set by control unit 175. In some examples, a user may select a target image brightness, and in other examples control unit 175 may automatically determine a target image brightness. For example, control unit 175 may use prior procedure data to determine a target image brightness to use in method 200. In some examples, the target image brightness may be a range of brightness values to apply to the one or more illuminators 127-130, ranging from a target low (T_{low}) brightness value to a target high (T_{high}) brightness value. For example, the range of brightness values used for the target image brightness may be a tolerance band set around a particular target brightness value. Also during step 201, the user or control unit 175 may set an initial illumination value to apply to the one or more illuminators 127-130.

In the next step 202, control unit 175 may determine an actual illumination value by accumulating and summing the value of the pixels in the current image frame received from one or more imaging devices 125, 126. In some examples, only a single image frame from a single imaging device 125, 126 may be used to determine the initial illumination value, and in other examples a plurality of image frames may be used from one or more imaging devices 125, 126.

During the time between frames received from the one or more imaging devices 125, 126 (e.g. during the vertical blanking), step 203 includes determining the error and PID coefficients for the current image frame. To determine the error coefficient for the current image frame, the control unit may execute the following calculation:

Error=T_{high}−B_{Current} when B_{Current}>T_{high}

Error=T_{low}—B_{Current} when B_{Current}<T_{low}

B_{Current} is the calculated illumination value of the current image frame. T_{low} is the target low illumination value, and T_{high} is the target high illumination value. In some examples, control unit 175 may determine the target range of brightness (or range of illumination values) to be a tolerance band around a target illumination value set by the user. To determine the PID coefficients, control unit 175 may execute the following calculations:

P=K_{P}*(Error)

I=K_{I}*(Error+ErrorOld)

D=K_{D}*(Error−ErrorOld)

Then ErrorOld=(Error)

K_{P} is the proportional tuning constant. K_{I} is the integral tuning constant, and K_{D} is the derivative tuning constant. Error is the calculated error coefficient for the current image frame, and ErrorOld is the calculated error coefficient of the immediately prior image frame. The tuning parameters (e.g. tuning constants K_{P}, K_{I}, and K_{D}) are determined experimentally and are dependent on the type of illumination used, as well as the driving circuit. In some examples, tuning constants K_{P}, K_{I}, and K_{D} may be experimentally determined to achieve a targeted response time. The speed of the PID loop is directly related to the tuning constants K_{P}, K_{I}, and K_{D}. Tuning constant K_{P} adjusts the output in proportion to the current error. Tuning constant K_{I} controls the static error. Tuning constant K_{D} is based on the rate of change of the error and provides a damping effect on the output.

In step 204, once the error and PID coefficients for the current image frame are determined using the above-described calculations, control unit 175 may determine a new illumination value to apply to the one or more illuminators 127-130, for example, using the old illumination value, the PID coefficients, and a hardware scaling factor. The new illumination value may be determined the following calculation:

Temporary_Value=(P+I+D)/F_{Scaling}

B_{New}=B_{Current}+Temporary_Value

Then B_{Current}=B_{New}

F_{Scaling} is a scaling factor based on the hardware used to drive the illumination, such as the type of illuminator (LED, fiber optic, etc.) and circuitry connected to the illuminator. Scaling factor F_{Scaling} may be based on the driving circuit, and may depend on the specific hardware implementation and the desired range of allowable illumination. Temporary_Value is an intermediate value used by the control unit to determine B_{New}, or a new illumination value to apply to the one or more illuminators 127-130. Note the calculated illumination values are digital numbers that are converted to an analog voltage, current, or any other digital value controlling illumination applied to the one or more illuminators 127-130, thus allowing control unit 175 to control the illumination of endoscope 101. In some examples, control unit 175 will repeat steps 202-204 for the next image frame from the one or more imaging devices 125, 126 once step 204 is completed and B_{New} is applied to the one or more illuminators 127-130. Thus, steps 201-204 are an example of a control loop algorithm to automatically adjust the illumination of endoscope system 100.

In some examples, the control loop algorithm of FIG. 2 may include an additional step 205 of determining if B_{New}, the new illumination value, is near the top or bottom of the range of illumination values accepted by a particular illuminator, or the range of illumination values the illuminator's hardware is able to accept. In some examples, the control loop algorithm may not include step 205 and may proceed from step 204 to step 202 (shown in dotted lines) to continue cycling through the loop algorithm.

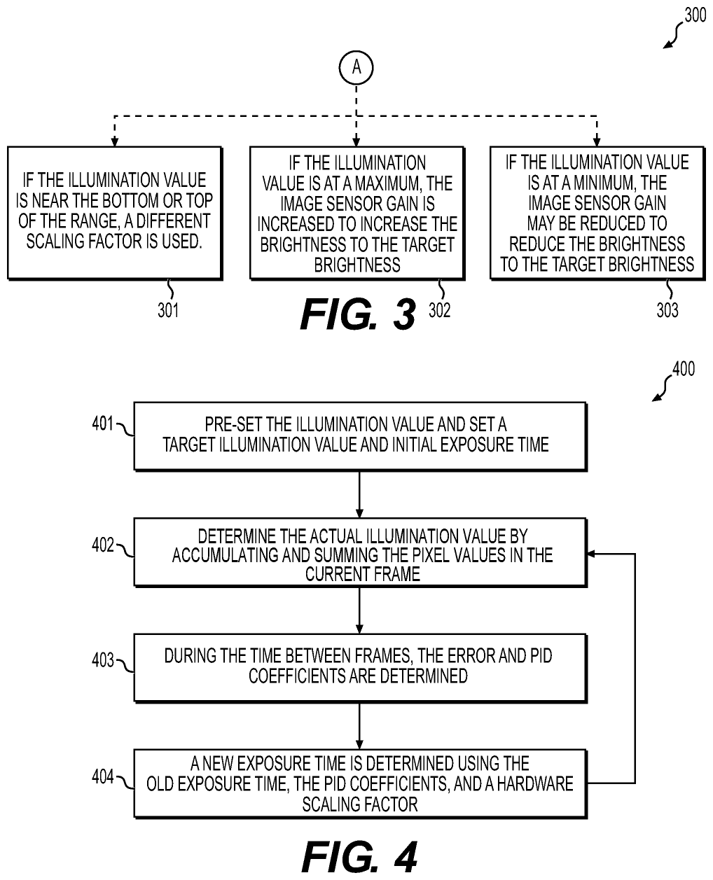

FIG. 3 illustrates the different steps 301-303 that control unit 175 may execute if the B_{New} is near the bottom or top of the range of illumination values accepted by the one or more illuminators, if the B_{New} is at the maximum of the range of illumination values accepted by the one or more illuminators, and if the B_{New} is at the minimum of the range of illumination values accepted by the one or more illuminators, respectively.

In step 301, if the B_{New} is near the bottom or top of the range of illumination values accepted by the one or more illuminators, a different scaling factor (F_{Scaling}) is used during the next cycle of the algorithm of FIG. 2. This different scaling factor (F_{Scaling}) is smaller than the previously used scaling factor (F_{Scaling}) to force the change in the illumination value to be smaller than the previous change in illumination value. Adjusting the scaling factor (F_{Scaling}) when B_{New} is near the bottom or top of the range of illumination values may facilitate the reduction or elimination of any oscillation in the lighting caused by nonlinearities in the hardware of the one or more illuminators.

In step 302, B_{New} is at the maximum of the range of illumination values accepted by the one or more illuminators (e.g. the illumination value is at a maximum). Since the illumination value cannot be increased beyond the maximum of the range of illumination values accepted by the one or more illuminators, control unit 175 may adjust the digital gain of the one or more image sensors associated with the one or more imaging devices 125, 126. For example, under normal conditions where B_{New} is at the maximum of the range of illumination values and the illumination or brightness level of the current image frame is below a minimum target illumination value, the gain of the one or more image sensors of the one or more imaging devices 125, 126 is increased. The gain is then increased (e.g., in a step-wise manner) until the minimum target illumination value for the current image frame is reached or the maximum gain of the one or more image sensors is reached. In some examples, if a saturation value (e.g., a value representing an intensity of color) for the current image frame falls from a target saturation value as the gain of the one or more image sensors is being increased in the step-wise manner, the gain may instead be increased to the maximum gain, while the illumination value for the current image frame is reduced.

In step 303, B_{New} is at the minimum of the range of illumination values accepted by the one or more illuminators (e.g. the illumination value is at a minimum). Since the illumination value cannot be decreased beyond the minimum of the range of illumination values accepted by the one or more illuminators, control unit 175 may adjust the digital gain of the one or more image sensors associated with the one or more imaging devices 125, 126. For example, under normal conditions where B_{New} is at the minimum of the range of illumination values and the illumination or brightness level of the current image frame is above a maximum target illumination value, the gain of the one or more image sensors of the one or more imaging devices 125, 126 is decreased. The gain is then decreased (e.g., in a step-wise manner) until the maximum target illumination value for the current image frame is reached or the minimum gain of the one or more image sensors is reached. In some examples, if a saturation value for the current image frame falls from a target saturation value as the gain of the one or more image sensors is being decreased in the step-wise manner, the gain may instead be decreased to the minimum gain, while the illumination value for the current image frame is increased. After completing any of steps 301, 302, or 303, control unit may continue with another cycle of the control loop algorithm of FIG. 2, for example, starting with step 202 of determining the actual image brightness for the next image frame received from the one or more imaging devices 125, 126. When the control loop algorithm executed by control unit 175 incorporates steps 205 and 301-303, control unit 175 may automatically switch between (i) adjusting the illumination value applied to the one or more illuminators 127-130 and (ii) adjusting the gain of the one or more image sensors of the one or more imaging devices 125, 126.

In some examples, to speed up the response of control unit 175 to extreme differences between B_{Current} and a target illumination value, an extreme image brightness guard may be used to change the gain of the one or more image sensors by values greater than 1. The extreme image brightness guard may be a minimum difference between B_{Current} and the target illumination value. Once the extreme image brightness guard is met (or the minimum difference between B_{Current} and the target illumination level is met), the gain will be increased by control unit 175 by a value proportional to the difference between B_{Current} and the target illumination value. For example, if the gain is in the low end of the range of gain values, and the B_{Current} suddenly drops well below the target illumination value, the gain is increased by control unit 175 by a value proportional to the difference between B_{Current} and the target illumination value. This scaling factor may be 2, 5, 10, or any number appropriate to more quickly reach the target illumination value. By adjusting the rate at which the gain is increased or decreased, the lag time to reach the target illumination value is decreased. When the extreme image brightness guard is not reached, the gain is increased or reduced by 1 as needed to reach the target illumination value.

FIG. 4 illustrates another illumination control method 400 that may be automatically executed by control unit 175 of endoscope system 100 (or another control unit). Method 400 of FIG. 4 utilizes proportional, integral, and derivative (PID) coefficients to control the speed and accuracy of the illumination in endoscope system 100. At initial step 401, a target image brightness (e.g. a target illumination value) is stored in control unit 175, and an initial illumination value of the one or more illuminators 127-130 (e.g. a pre-set illumination value) and an initial exposure time of the one or more imaging devices 125, 126 is determined by control unit 175. The initial illumination value applied by control unit 175 in method 400 may be a specific illumination value that provides enough light such that the image brightness is adequate for average imaging volumes, at the maximum exposure available in imaging devices 125, 126 for the desired frame rate. This specific illumination value may be set by a user or automatically applied by control unit 175. For example, default illumination values may achieve approximately 40-50% image brightness on average, and a user may be able to adjust the illumination value in the range between 25-70% image brightness, based on the user preference and clinical need. In some examples, a user may select a target image brightness (e.g. target illumination value), and in other examples control unit 175 may automatically determine a target image brightness. For example, control unit 175 may use prior procedure data to determine a target image brightness to use in the method of FIG. 2. In some examples, the target image brightness may be a range of brightness values to apply to the one or more illuminators 127-130, ranging from a target low (T_{low}) brightness value to a target high (T_{high}) brightness value.

In the next step 402, in the same manner as described hereinabove in relation to step 202 of FIG. 2, control unit 175 may accumulate and sum the value of the pixels in the current image frame received from one or more imaging devices 125, 126. In some examples, only a single image frame from a single imaging device 125, 126 may be used to determine the initial illumination value, and in other examples a plurality of image frames may be used from one or more imaging devices 125, 126.

During the time between frames received from the one or more imaging devices 125, 126 (e.g. during the vertical blanking), step 403 includes determining the error and PID coefficients for the current image frame. The error and PID coefficients are determined in the same manner as the method described above in relation to FIG. 2. F_{Scaling} may change when using the PID loop with exposure time, for example, a user may want to scale the output to have smaller steps to more finely control the illumination. Once the error and PID coefficients for the current image frame are determined, control unit 175 may determine a new illumination value and, in step 404, adjust the exposure time based on the new illumination value. For example, the exposure time is reduced when the image is too bright (e.g. B_{New}>B_{Current}), and the exposure time is increased when the image is too dim (e.g. B_{Current}>B_{Newt}). The exposure time may be increased by a single unit, or by plurality of units, after each execution of the PID algorithm loop for the current frame. Once the exposure time reaches a maximum value and the image remains too dim (e.g. B_{Current}>B_{Newt}), the illuminator value provided to the one or more illuminators 127-130 may be increased using the same illuminator control algorithm described hereinabove in relation to FIG. 2.

Sensor manufacturers typically have controllable registers for exposure such that the number written to the register of registers is in some fraction of a line. For example, a sensor with 480 lines running at 30 frames per second will have a line time of approximately 65 microseconds. If the exposure number in the register corresponds to 1/16 of a line, then writing the number 16 to the register would give an exposure time of approximately 65 microseconds. The maximum exposure allowed for a particular image sensor, in this example, would be approximately 30 microseconds, since any exposure longer than 30 microseconds will force the frame rate to decrease. By knowing the exposure in fractions of a line, the exposure of an image sensor may be controlled using a PID based algorithm, such as method 400 of FIG. 4. By adjusting the exposure time of the one or more imaging devices 125, 126 and adjusting the illumination value applied to the one or more illuminators 127-130 using method 400 of FIG. 4, the spectral stability of the light used for illuminating target anatomy may be increased. In addition, method 400 may facilitate minimizing color shifts due to different lighting scenarios.

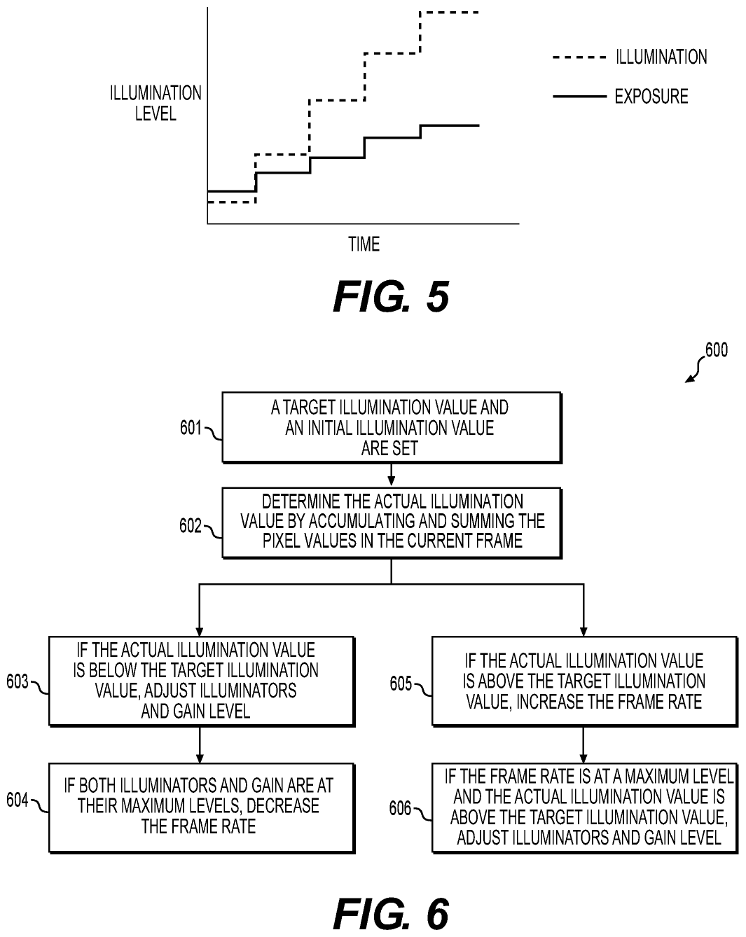

FIG. 5 illustrates an exemplary chart of the illumination values applied to one or more illuminators 127-130 in a PID control system, such as the system described in relation to FIG. 2, and the exposure time values applied to one or more imaging devices 125, 126 in a PID control system, such as the system described in relation to FIG. 4. By using the slopes of the illumination values and the exposure time values, a more complex method may be utilized to adjust the brightness of an image that switches between (i) adjusting the illumination values of one or more illuminators 127-130 and (ii) adjusting the exposure time of the one or more imaging devices 125, 126. For example, if the demand for a change in brightness is large (e.g. above a certain threshold value), the illumination value applied to the illuminators 127-130 can be adjusted to modify the image brightness. By adjusting the illumination value applied to the illuminators 127-130, the increase or decrease in brightness will take effect in less time than if the exposure time was adjusted. If the demand for a change in brightness is small (e.g. below a certain threshold value), the exposure time of the one or more imaging devices 125, 126 can be adjusted to modify the image brightness. The increase or decrease in brightness as a result of a single step up or down in the exposure time will have a smaller effect on the image brightness than a single step up or down in the illumination value. By toggling between adjusting the illumination value applied to illuminators 127-130 and adjusting the exposure time applied to the imaging devices 125, 126, the brightness of an image may be controlled with greater precision, particularly near the low limits of the illumination control.

FIG. 6 illustrates another illumination control method 600 that may be automatically executed by control unit 175 of endoscope system 100 (or another control unit). Method 600 of FIG. 6 utilizes proportional, integral, and derivative (PID) coefficients to control the speed and accuracy of the illumination in endoscope system 100. Method 600 of FIG. 6 incorporates control of the frame rate of imaging devices 125, 126 as an additional aspect of controlling brightness of a received image, in combination with any of the other methods discussed hereinabove in relation to FIGS. 2-5. As shown in FIG. 6, at initial step 601, a target image brightness (e.g. a target illumination value) is set (e.g., stored in control unit 175), and an initial illumination value of the one or more illuminators 127-130 is determined by control unit 175. Also during step 601, an initial frame rate is set (e.g., stored in control unit 175) at the one or more imaging devices 125, 126.

In the next step 602, in the same manner as described hereinabove in relation to step 202 of FIG. 2, control unit 175 may determine actual image brightness by accumulating and summing the value of the pixels in the current image frame received from one or more imaging devices 125, 126.

During the time between frames received from the one or more imaging devices 125, 126 (e.g. during the vertical blanking), at steps 603 and 605, control unit 175 determines whether the actual illumination value of the current image frame is below (step 603) or above (step 605) the target illumination value.

If the actual illumination value of the current image frame is below (step 603) the target illumination value, control unit 175 will (i) first adjust the illumination value applied to the one or more illuminators 127-130 until either the maximum illumination value for the one or more illuminators 127-130 is reached or the target illumination value is reached, and then (ii) adjust the gain value applied to the one or more imaging devices 125, 126 until either the maximum gain value is reached or the target illumination value is reached. As shown in step 604, if both the illumination value applied to the one or more illuminators and the gain value applied to the one or more imaging devices 125, 126 are at their respective maximum values, control unit 175 will proceed to decrease the frame rate of the one or more imaging devices 125, 126, to allow for an increase in exposure time, until either the target illumination value is reached for the received image or the minimum frame rate is reached.

If the actual illumination value of the current image frame is above (step 605) the target illumination value, control unit 175 will increase the frame rate applied to the one or more imaging devices 125, 126 until either the target illumination value is reached for the received image or the maximum frame rate is reached. Then, if the actual illumination value is still above the target illumination value and the maximum frame rate is reached (step 606), control unit 175 will then (i) adjust the illumination value applied to the one or more illuminators 127-130 until either the minimum illumination value for the one or more illuminators 127-130 is reached or the target illumination value is reached, and then (ii) adjust the gain value applied to the one or more imaging devices 125, 126 until either the minimum gain value is reached or the target illumination value is reached. By combining the automatic adjustment of the illumination value applied to the one or more illuminators 127-130, the gain value applied to the one or more imaging devices 125, 126, and the frame rate applied to the one or more imaging devices 125, 126, the image brightness may more efficiently be adjusted, for example, to minimize color shifts due to different lighting scenarios. A higher frame rate may also lead to a reduced video latency, as well as allowing for the appearance of a “smoother” video.

In various embodiments, any of the systems and methods described herein may include control unit 175 and a medical device (e.g., endoscope 101), and control unit 175 may include a processor, in the form of one or more processors or central processing unit (“CPU”), for executing program instructions. In some examples, the one or more processors may be one or more processing boards. Control unit 175 may include an internal communication bus, and a storage unit (such as ROM, HDD, SDD, etc.) that may store data on a computer readable medium, although control unit 175 may receive programming and data via network communications. Control unit 175 may also have a memory (such as RAM) storing instructions for executing techniques presented herein, although the instructions may be stored temporarily or permanently within other modules of control unit 175 (e.g., processor and/or computer readable medium) or remotely, such as on a cloud server electronically connected with control unit 175. The various system functions of control unit 175 may be implemented in a distributed fashion on a number of similar platforms, to distribute the processing load. Alternatively, the systems discussed herein may be implemented by appropriate programming of one computer hardware platform at control unit 175.

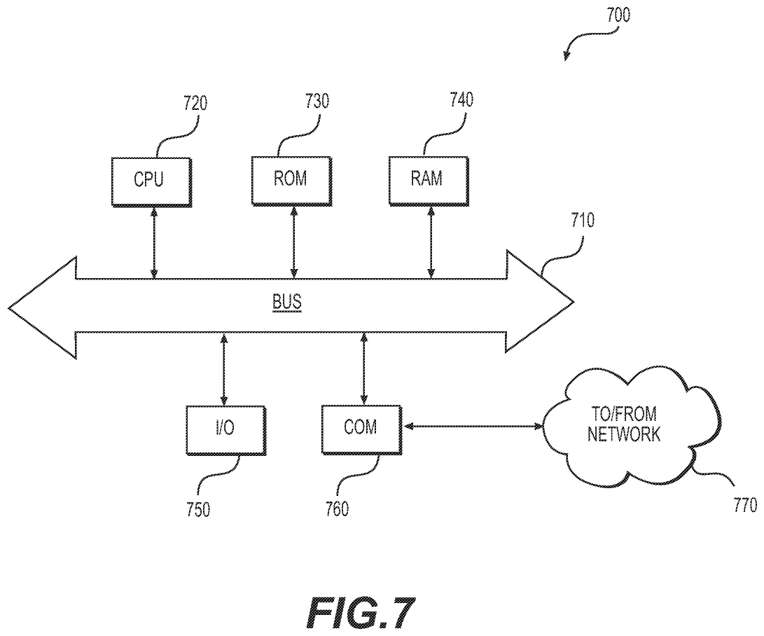

FIG. 7 provides a functional block diagram illustration of general purpose computer hardware platforms. FIG. 7 illustrates a network or host computer platform, as may typically be used to implement a server 700 or a browser, or any other device executing features of the methods and systems described herein. It is believed that those skilled in the art are familiar with the structure, programming, and general operation of such computer equipment and as a result, the drawings should be self-explanatory.

A platform for the server 700 or the like, for example, may include a data communication interface for packet data communication 760. The platform may also include a central processing unit (CPU) 720, in the form of one or more processors, for executing program instructions. The platform typically includes an internal communication bus 710, program storage, and data storage for various data files to be processed and/or communicated by the platform such as ROM 730 and RAM 740, although the server 700 often receives programming and data via network communications 770. The hardware elements, operating systems, and programming languages of such equipment are conventional in nature, and it is presumed that those skilled in the art are adequately familiar therewith. The server 700 also may include input and output ports 750 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology discussed herein may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to control unit 175, it should be appreciated that the disclosed embodiments may be applicable to any environment, such as a desktop or laptop computer, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among steps shown in the figures. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

We claim:

1. A medical device system comprising:
a control unit configured to be operatively coupled to a medical device, wherein the control unit comprises:
one or more processors that implement an algorithm to enhance images obtained by a first viewing element of the medical device, wherein the one or more processors perform the steps of:
receiving a first image from the first viewing element;
determining a current illumination value of the first image;
determining a first difference between the current illumination value and a target high illumination value if the current illumination value is greater than the target high illumination value;
determining a second difference between the current illumination value and a target low illumination value if the current illumination value is less than the target low illumination value;
generating a new illumination value, using at least one of the first difference and the second difference; and
converting the new illumination value to a first voltage value for application to one or more illuminators of the medical device.

2. The system of claim 1, wherein the target high illumination value and the target low illumination value together define a tolerance band around a target illumination value stored by the control unit.

3. The system of claim 1, wherein the one or more processors further perform the steps of:
determining if the current illumination value is below a first threshold illumination value; and
if the current illumination value is below the first threshold illumination value, using a scaling factor to generate the new illumination value.

4. The system of claim 1, wherein the one or more processors further perform the steps of:
determining if the new illumination value is greater than a maximum illumination value; and
if the new illumination value is greater than a maximum illumination value, converting the maximum illumination value to a second voltage value for application to one or more illuminators of the medical device, and increasing a gain of one or more imaging devices of the medical device.

5. The system of claim 1, wherein the one or more processors further perform the steps of:
determining if the new illumination value is lower than the current illumination value; and
if the new illumination value is lower than the current illumination value, decreasing a gain of one or more imaging devices of the medical device.

6. The system of claim 1, wherein the medical device is an endoscope.

7. The system of claim 1, wherein determining the current illumination value of the first image includes accumulating and summing pixel values of the first image.

8. The system of claim 1, wherein the one or more processors further perform the steps of:
determining a current frame rate of the first viewing element;
generating a new frame rate, using at least one of the first difference and the second difference; and
applying the new frame rate to the first viewing element.

9. The system of claim 6, further comprising the medical device including the first viewing element and at least one illuminator.

10. The system of claim 1, wherein the one or more processors further perform the steps of:
determining a current exposure time of the first viewing element;
generating a new exposure time, using at least one of the first difference and the second difference; and
applying the new exposure time to the first viewing element.

11. The system of claim 1, wherein the one or more processors further perform the steps of:
prior to converting the new illumination value to the first voltage value, determining if the current illumination value is below or above the new illumination value;
if the current illumination value is below the new illumination value, converting the new illumination value to the first voltage value; and
if the current illumination value is above the new illumination value, increasing a frame rate of the first viewing element.

12. The system of claim 1, wherein generating a new illumination value, using at least one of the first difference and the second difference, includes determining a first error coefficient of the first image and a second error coefficient of a second image, wherein the second image was received by the control unit prior to the first image;
wherein the first error coefficient is the first difference if the current illumination value is greater than the target high illumination value; and
wherein the first error coefficient is the second difference if the current illumination value is less than the target low illumination value.

13. The system of claim 12, wherein generating the new illumination value further includes determining a proportional tuning constant, an integral tuning constant, and a derivative tuning constant each associated with the medical device.

14. The system of claim 1, wherein the current illumination value is a first current illumination value, the target high illumination value is a first target high illumination value, the target low illumination value is a first target low illumination value, and the new illumination value is a first new illumination value, and
wherein the one or more processors further perform the steps of:
receiving a second image from a second viewing element;
determining a second current illumination value of the second image;
determining a third difference between the second current illumination value and a second target high illumination value if the second current illumination value is greater than the second target high illumination value;
determining a fourth difference between the second current illumination value and a second target low illumination value if the second current illumination value is less than the second target low illumination value;
generating a second new illumination value, using at least one of the third difference and the fourth difference; and
converting the second new illumination value to a second voltage value for application to one or more illuminators of the medical device.

15. The system of claim 1, wherein the one or more processors further perform the steps of:
displaying, via at least one electronic display, a second image received from the first viewing element, wherein the second image is illuminated by the one or more illuminators receiving the first voltage value.

16. A method of enhancing images obtained by a medical device system, wherein the medical device system comprises (a) one or more processers, and (b) a medical device operatively coupled to the one or more processers, wherein the medical device is configured to be inserted into a body of a patient and includes a first viewing element and one or more illuminators, the method comprising the steps of:
receiving a first image from the first viewing element;
determining a current illumination value of the first image;
determining a first difference between the current illumination value and a target high illumination value if the current illumination value is greater than the target high illumination value;
determining a second difference between the current illumination value and a target low illumination value if the current illumination value is less than the target low illumination value;
generating a new illumination value, using at least one of the first difference and the second difference; and
converting the new illumination value to a first voltage value for application to the one or more illuminators of the medical device.

17. The method of claim 16, further comprising the steps of:
determining if the new illumination value is greater than a maximum illumination value; and
if the new illumination value is greater than a maximum illumination value, converting the maximum illumination value to a second voltage value for application to one or more illuminators of the medical device, and increasing a gain of one or more imaging devices of the medical device.

18. The method of claim 16, further comprising the steps of:
determining a current exposure time of the first viewing element;
generating a new exposure time, using at least one of the first difference and the second difference; and
applying the new exposure time to the first viewing element.

19. The method of claim 16, further comprising the steps of:
determining a current frame rate of the first viewing element;
generating a new frame rate, using at least one of the first difference and the second difference; and
applying the new frame rate to the first viewing element.

20. A non-transitory computer readable medium containing program instructions for causing a computer to perform a method of enhancing images obtained by a first viewing element in a medical device system, wherein the medical device system comprises a processor configured to implement the method, and a medical device operatively coupled to the processor, the medical device being configured for insertion into a body of a patient and including the first viewing element and one or more illuminators, the method comprising the steps of:
receiving a first image from the first viewing element;
determining a current illumination value of the first image;
determining a first difference between the current illumination value and a target high illumination value if the current illumination value is greater than the target high illumination value;
determining a second difference between the current illumination value and a target low illumination value if the current illumination value is less than the target low illumination value;
generating a new illumination value, using at least one of the first difference and the second difference; and
converting the new illumination value to a first voltage value for application to the one or more illuminators of the medical device.
